(19) United States
(12) Patent Application Publication
(10) Pub. No.: US 2017/0036009 A1
(43) Pub. Date: Feb. 9, 2017

(54) TENS with vibration and/or mechanical muscular manipulation

(71) Applicants: Clint Hughes, Plano, TX (US); Theodore Maltezos, Dallas, TX (US); George Sadler, Hunt Valley, MD (US)

(72) Inventors: Clint Hughes, Plano, TX (US); Theodore Maltezos, Dallas, TX (US); George Sadler, Hunt Valley, MD (US)

(21) Appl. No.: 14/816,649

(22) Filed: Aug. 3, 2015

(51) Int. Cl. A61N 1/04; A61H 23/02; A61H 7/00; A61N 1/22

(52) U.S. Cl. CPC A61N 1/0456; A61N 1/0492; A61N 1/22; A61H 23/0254; A61H 7/004; A61H 2201/10

(57) ABSTRACT

A TENS (transcutaneous electrical nerve stimulation) device and method using a micro current with a carrier signal and a square wave form combined with vibration and/or mechanical muscular manipulation for promoting the release and disbursement of lactic acid treating muscular soreness, cramping or degeneration. This method and nerve stimulation device is packaged to require no input from a user and a user must only apply the sticky electrode pad with the base unit attached on top to the correct part of the body and start the preprogrammed sequence of electrical currents, vibration and/or muscular manipulation. The method involves applying bursts of direct current at higher frequencies for shorter periods of time followed by lower frequency bursts of electrical current for longer periods of time combined with a small vibration motor and/or motorized nodes providing muscular manipulation to certain areas of the body.

This invention relates generally to a TENS (transcutaneous electrical nerve stimulation) apparatus for use for therapeutic purposes. This device proposes using electrical current in the micro amp range (less than one milliamp) for specific wave forms and carrier waves combined with vibration and/or mechanical muscular manipulation. The treatment duration is of a definite length and sequence with proposed automatic controls. The wave form, the current, and the duration of treatment are all designed to maximize therapeutic benefits. Micro current is defined as current below one milliamp. For pain treatment, it is believed that the micro current blocks neural transmission of pain signals and stimulates release of endorphins known to be naturally occurring pain relieving chemicals. This combined with vibration and/or mechanical muscular manipulation temporarily increases blood flow to an area and provides muscle stimulation. The combination of the blocking of the pain signals and release of the endorphins provides for relief of both chronic and acute pain. It is also believed that micro current stimulation can promote healing. It is believed that this can be done by increasing blood vessel permeability, increasing adenosine triphosphate levels, or restoring cellular electrical balances by changing electric potentials across cell membranes.

One TENS device is described in the Rossen U.S. Pat. No. 4,989,605. It is believed this device has been sold on the market by the trade name “MicroStim”. The Rossen '605 patent discloses a micro current TENS unit that uses a unique wave form. It is proposed the current is from 250 micro amps up to about 900 micro amps with a peak current of six milliamps. The current is applied through a pair of electrodes in the form of high-frequency monophasic bursts of a direct current with a carrier signal from around 10,000 Hz to 19,000 Hz. The signal is modulated at a relatively lower frequency (0.3 Hz up to 10,000 Hz). These modulated carrier signals are from about 0.05 seconds to 10 seconds in duration with above one second being the preferred duration. The electrodes are reversed as simulating a biphasic form yet the character is a monophasic DC signal. The Rossen patent is for palliative pain treatment only.

The Wallace U.S. Pat. No. 5,522,864 proposes that macular degeneration or other ocular pathology may be treated by placing a positive electrode of a direct micro current source in contact with the closed eyelid of the subject and placing a negative electrode away from the eye of the subject, preferably on the neck of the subject. These electrodes apply a constant direct current of 200 micro amps for approximately 10 minutes. It is proposed that this device can be portable and battery powered, hence allowing a subject undergoing the treatment to ambulate during the treatment. The Wallace patent proposes using micro current to treat macular degeneration but does not disclose the mechanism by which positive results are obtained.

The Jarding et al. U.S. Pat. No. 6,275,735 proposes digital control of the modulation frequency of the micro current signal. The modulation frequency is controlled by a digital data word. A controller is coupled to a digital analog converter and supplies the digital analog converter with digital data words to generate an electrical signal for the micro current stimulation therapy. It is believed that this form of micro current therapy may be particularly useful in macular degeneration. More specifically, the Jarding patent proposes that adenosine triphosphate levels in cells can be affected by appropriate electrical stimulation. Jarding proposes that electrical stimulation to the cells increases blood vessel permeability, increasing ATP levels and increasing protein synthesis. Therefore, Jarding concludes that micro current stimulation can help rejuvenate cells in the retina to slow or stop degeneration of the eye due to age-related macular degeneration. Therefore, Jarding proposes a computer controlled electrical stimulation to maximize therapeutic benefits by varying the types of wave forms and frequency ranges used in the therapy.

Another problem present in the use of TENS units for therapeutic or palliative effect is patient compliance. Wingrove U.S. Pat. No. 5,800,458 proposes a compliance monitor to determine if the patient is using the TENS unit in accordance with instructions or prescriptions. Another problem with conventional TENS units is they can be bulky or difficult to control. This can especially apply to individuals who have some disability or who are in acute pain. Michelson et al. U.S. Pat. No. 6,445,955 proposes a miniature wireless TENS unit. A remote controller sends transmission signals to a receiver within an electronic module with the TENS unit allowing the patient to program specific units in a specific way. The TENS unit itself may be incorporated within a bandage or electrode package and worn directly on the patient's body.

It has also been proposed, at least in a research context, that the wrong current levels used in transcutaneous electrical nerve stimulation can actually reduce ATP levels. Research by Ngok Chen would demonstrate, at least in rats, that current levels in the micro amp range tended to increase ATP concentration in cells while currents in the milliamp range tended to lower ATP concentration in cells. (See Chen, The Effects of Electrical Current on ATP Generation, Protein Synthesis, and Membrane Transport in Rat Skin, Clinical Orthopedics Research, 171, November-December 1982, pp. 264-271) It has also been suggested by Thomas W. Wing, D. C., N. D. that direct current employing a carrier wave with a low frequency is very helpful triggering the repair process in muscles. Wing suggested only very low levels of stimulation are required if the effect of the direct electrical current at a low frequency was the triggering of the body's natural repair cycle. Wing suggests that low frequencies of 0.1 to 0.3 Hz produce lasting therapeutic effects, but pain relief is more rapid at higher frequencies in the 10 to 100 Hz range. Thomas W. Wing, D. C., N. D. Chiropractic Economics, March/April 1987. The Food and Drug Administration has approved the use of TENS units for symptomatic relief of chronic pain and to manage post surgical traumatic pain problems. The therapeutic use to treat macular degeneration proposed by Wallace et al in the '864 patent is an off label use of a TENS device, that is, this use is not approved by the FDA. However, in order to obtain FDA approval for a TENS unit for treatment of degenerative diseases such as macular eye disease, tissue repair, and cell regeneration, require proof of effectiveness. Proof is obtained through double blind, randomized, and multi-site clinical trials. It is believed such trials are underway to document effectiveness of a TENS unit for the treatment of age related macular degeneration.

The use of a TENS unit both for palliative and therapeutic treatment is well established, but the precise mechanism by which it operates is not fully understood. This means that even small differences in how an electrical current is applied can have unpredictably large changes in the therapeutic outcome. That is to say, a current of 200 micro amps might be therapeutic, while a current of 400 micro amps might actually be harmful. By the same token, such factors as whether the current is applied in a wave form, whether the polarity of the current alternates, and the like all can have important impacts in either the palliative or the therapeutic effect. Consequently, the optimal use of a TENS unit for either palliative or therapeutic effects proceeds more by experimentation than by theoretical design. That is to say, there is no theory by which one can design an ideal or maximally beneficial treatment modality for a particular TENS unit, then experiment to confirm the correctness of the program. Theory may point one in the right direction, but then an inventor must use intuition, clinical judgement, and experimentation to arrive at a therapeutic program giving maximum benefits.

Despite this earlier work there is still need for a TENS unit and treatment method to meet specific goals and needs. First, in the correct method, the TENS units must always operate in a current range that maximizes the benefits of the electrical nerve stimulation. Second, the duration and frequency of the electrical stimulation must be controlled to maximize the benefits. Third, control of the TENS unit should be designed to maximize compliance of a patient. Fourth, our device can combine TENS therapy, vibration therapy and/or mechanical muscular manipulation separately, paired together or all at the same time in one simultaneous treatment. Therefore, it is an object of the current invention to provide a fully automated and computer controlled micro current stimulation device with vibration and/or mechanical muscular manipulation for maximum effectiveness. It is a further object of the invention to administer a therapeutic electrical current in the micro amp range, always less than one milliamp. It is a further object of the invention that the current be administered in a square wave form in a sequential pattern of specific electrical bursts with frequencies between 0.1 Hz and 300 Hz. It is a further object of the invention to control the duration of the application of each electrical current bursts with the specified frequency for a specific period of time and sequence the application of the current controlled by an internal control to minimize patient compliance issues and to maximize benefit. It is a further object of this invention to periodically reverse the polarity of the current flow at specific intervals. It is a further object of this invention to provide vibration therapy and/or muscular manipulation in combination with the TENS therapy. It is an object of the invention to provide minimal controls or requirements for patient input or control.

### SUMMARY OF THE INVENTION

The present consists of a micro current stimulator ordinarily housed in a rigid casing. Inside that casing are controls to produce and apply a micro current transcutaneous electrical nerve stimulation and a small vibration motor. The controls are preprogrammed to provide a therapeutic level of electro current in the micro amperage range in a square wave form with a specific sequential pattern of specific electrical bursts with specific frequencies and to reverse the current flow at specific time intervals by reversing the electrode polarity. At the same time the electrical burst are occurring a vibration motor is spinning providing the area with mechanical vibration. In addition a third modality can be adapted to the device, mechanical manipulation. The unit can encompass a small spinning attachment that spins in a circular motion with nodes protruding out. This gives the user the ability to use all three modalities at the same time. The application of the specific electrical carrier frequencies and micro amperage current is controlled for duration and is sequenced by the control and the vibration is controlled as well. Ordinarily, there will be both visual and audible cues given to a user by the micro current stimulator. The multiple frequencies begin with a higher frequency and work down to a lower frequency while the vibration is at 1800 RPM's. This micro current stimulation unit has minimal settings adjustments or controls for patient use. A specific carrier frequency would be used in the range of 10,000 Hz to 15,000 Hz. The current will be delivered in the micro amp range less than one milliamp. A square wave form will be employed. Specific electrical bursts with specific frequencies, no more than 300 Hz and no less than 0.1 Hz, will be employed. A typical pattern of bursts would be a 200 Hz frequency applied for 180 seconds, a 15 Hz frequency applied for 240 seconds, a 6.1 Hz frequency applied for 360 seconds, and a 0.25 Hz applied for 720 seconds. The electrode polarity will be reversed every two seconds. During the entire runtime the vibration motor can be operating at 1800 RPM's and/or the small spinning attachment that spins in a circular motion with nodes protruding out, which ever combination provides the most relief to the user. A therapeutic program will ordinarily be in the range of 18 minutes of duration according to a pre-programmed, pre-set treatment sequence with controlled current levels and wave forms to have the greatest therapeutic value. The unit will have ways of alerting a user to malfunctions or to low-battery. Other features and advantages of the invention will become apparent in the Detailed Description of the Drawings, which follow.

### BRIEF DESCRIPTION OF THE DRAWINGS

FIG. 1 shows the micro current stimulation device.

FIG. 2 is a block diagram of the essential components of the micro current stimulation device.

FIG. 3 is a diagram of an embodiment of the current invention showing the sticky pads and the small vibration motor.

FIG. 4 illustrates a flowchart that demonstrates the method of the invention of applying a micro current across the electrodes using a direct current carrier signal having a frequency between 10,000 Hz and 20,000 Hz, which is modulated on and off at preset frequencies for preset periods of time in a sequence in which the preset frequencies sequentially decrease and the preset time periods sequentially increase.

FIG. 5 illustrates the embodiment attached to an arm.

FIG. 6 illustrates the embodiment with the strap to attach to the users arm.

FIG. 7 illustrates the embodiment with the strap attached to the users arm.

FIG. 8 illustrates a block diagram for the operations.

FIG. 9 illustrates the embodiment with the manual manipulation spinning part attached.

FIG. 10 illustrates the embodiment with the vibration separate and the base unit snaps into the embodiment.

FIG. 11 illustrates the embodiment which has the vibration built into it and the base unit snapped in attached to a users arm.

### DETAILED DESCRIPTION OF THE DRAWINGS

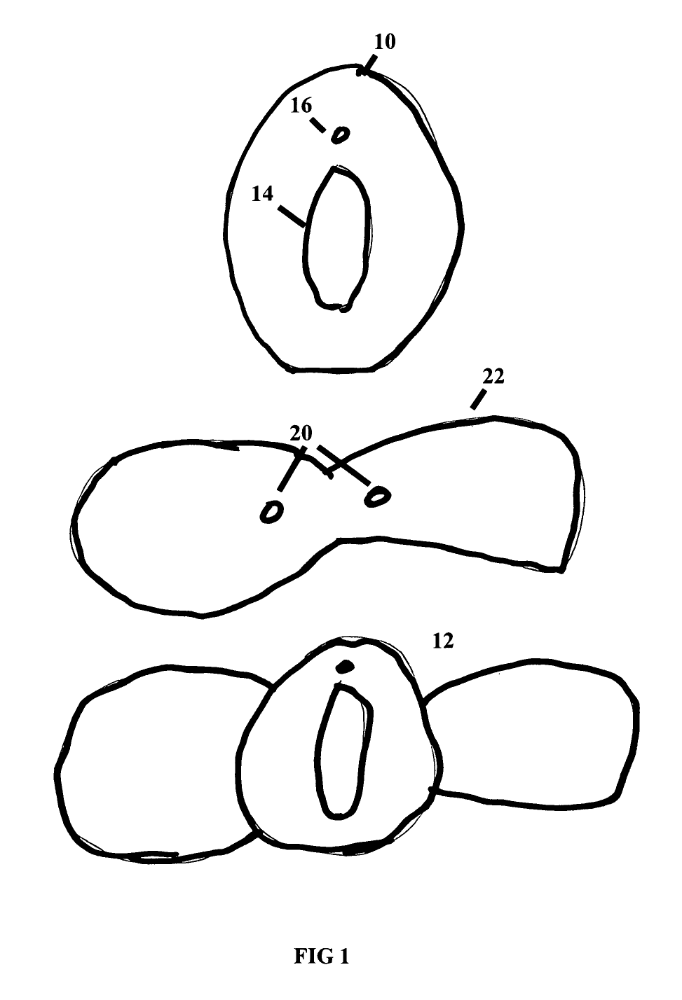

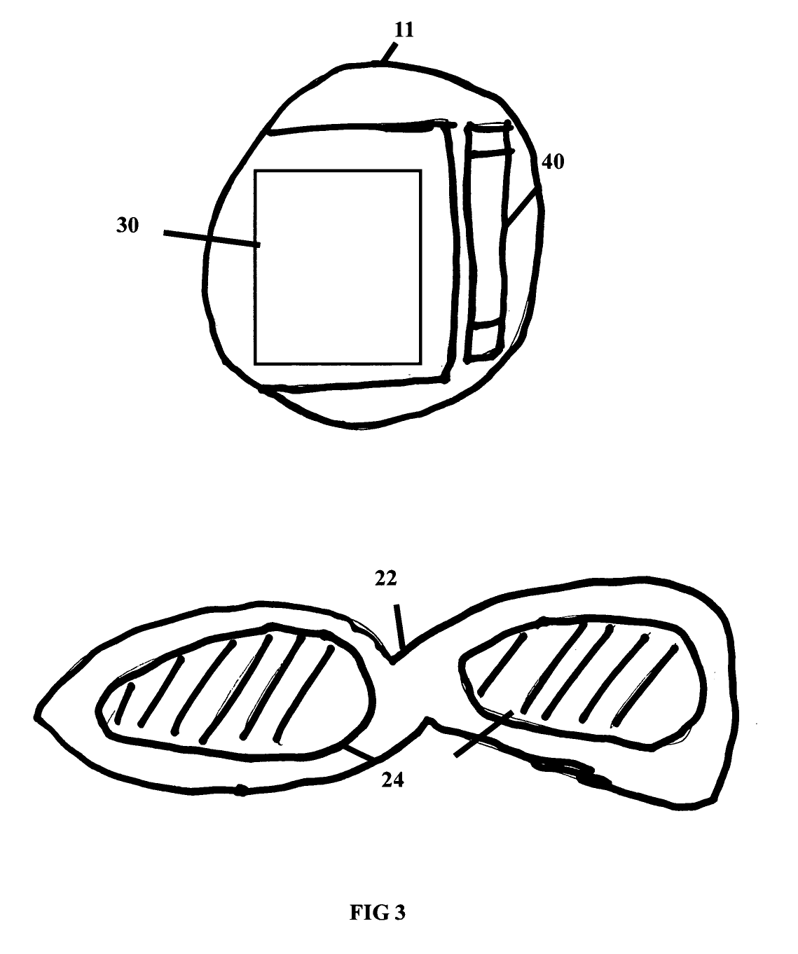

FIG. 1 shows the micro current stimulation device, base unit only (10). Inside there is a box (30) shown on FIG. 3 with various components and controls. Connected to the box (10) is an electrode pad (22) that connects to the box (10) as part of the micro current nerve stimulation device (10). If the polarity of the current is reversed as part of a treatment method, then more than two electrodes may be employed. Electrode (22) connects by means of snap pin (20) to electrode jacks located on the back of component (10). An on/off switch is (14) on the front of the box (10). Embodiment (16) is an illuminator led light which shows when the unit is operating for the user. Electrode (20) will be applied to a user completing an electrical circuit which allows a micro current to pass from one electrode through the body of the user to the other electrode to complete the circuit. Contained within the box (10) are control circuitry and microprocessors which may be programmed to provide particular types of current in particular wave forms, as will be explained later in the application. On the front of the box (12) show the unit attached to the electrode. Ordinarily, batteries will supply the power, which are contained in the unit.

To use the micro current nerve stimulation device (10), the user will ordinarily apply the electrode pad (22) to a prescribed place as determined by a health care provider. The on/off switch (14) will turn the device on to the appropriate amount of current. The micro current nerve stimulation device (10) will then begin to follow a pre-programmed sequence in which current will be provided with a particular frequency and wave form in the preferred program.

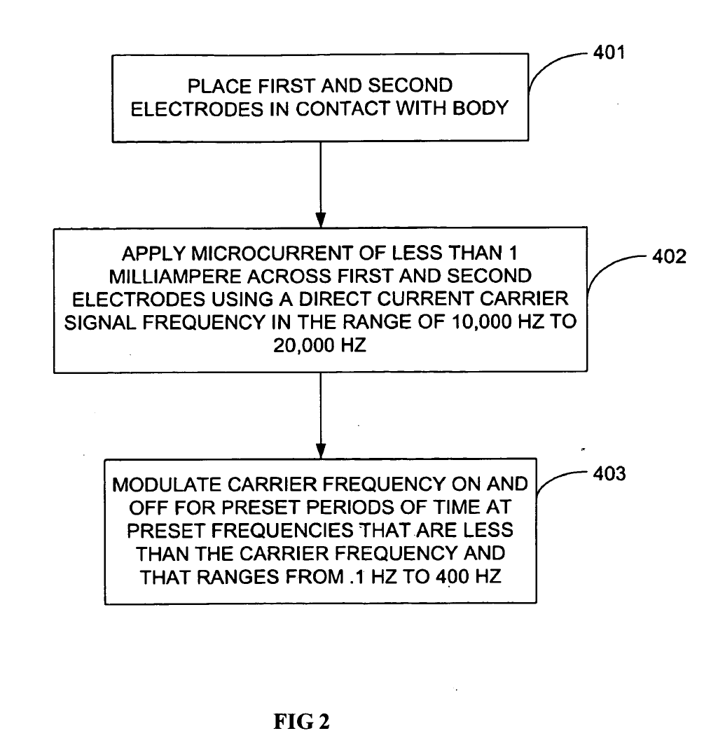

FIG. 2 is a block chart showing operating characteristics of the embodiment.

FIG. 3 shows the inside of the unit (30) the pc board parts and component that drive the units operations. The vibration unit is embodiment (40). The vibration (40) operates when switched on, the vibration unit will oscillate at 1800 RPM's. The electrode (22) is shown with the sticky side shown (24). The pad (22) has sticky conducting material adhered to the pad (22) on the back side and (24) sticks to the skin when unit is on or off and can be easily removed by hand.

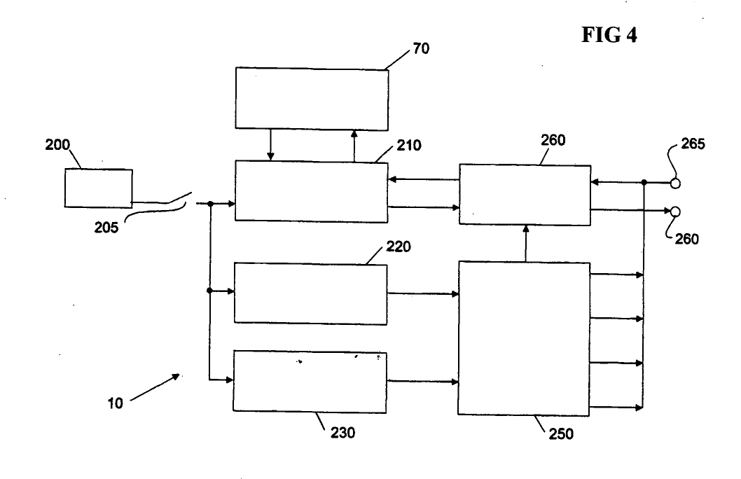

FIG. 4 current flows through an on/off switch (205) to voltage convertor (210) to a regulator (220) and to an oscillator (230). Current flows from the regulator (220) and oscillator (230) to a frequency divider (250). A constant current source (260) receives current from the frequency divider (250) and from the voltage convertor (210) then passes from the constant current source to a first electrode (260). A circuit is completed from the first electrode (260) through the patient (not shown) to a second electrode (265). Current returns to the constant current source (260) to the voltage convertor (210) through an amplitude control (70) and then to the power source (200) to complete the circuit.

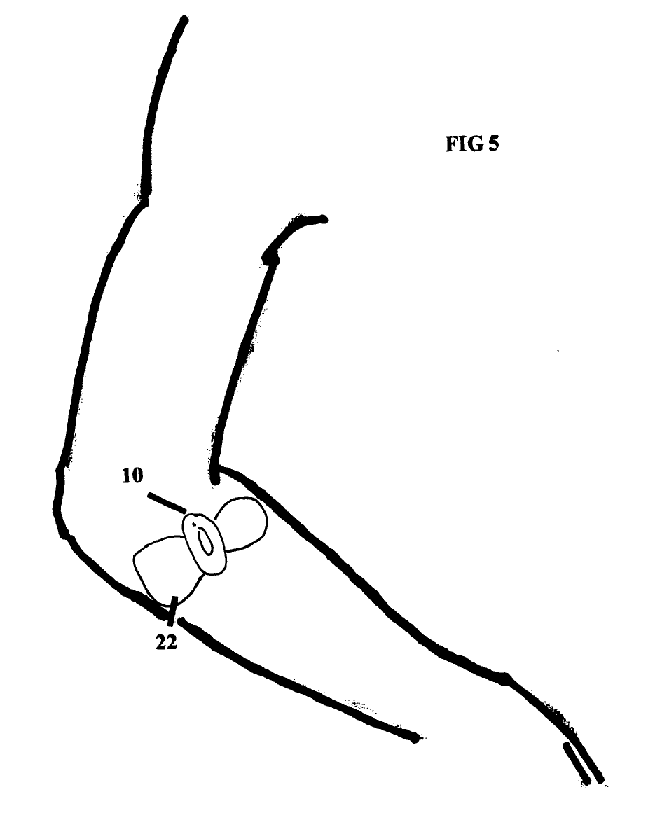

FIG. 5 is a diagram of an arm showing the unit (10) attached to someone's arm. FIG. 5 shows the embodiment attached to an arm not using the strap. Base unit (10) is snapped on (22) and sticky pad (22) is adhered to the skin.

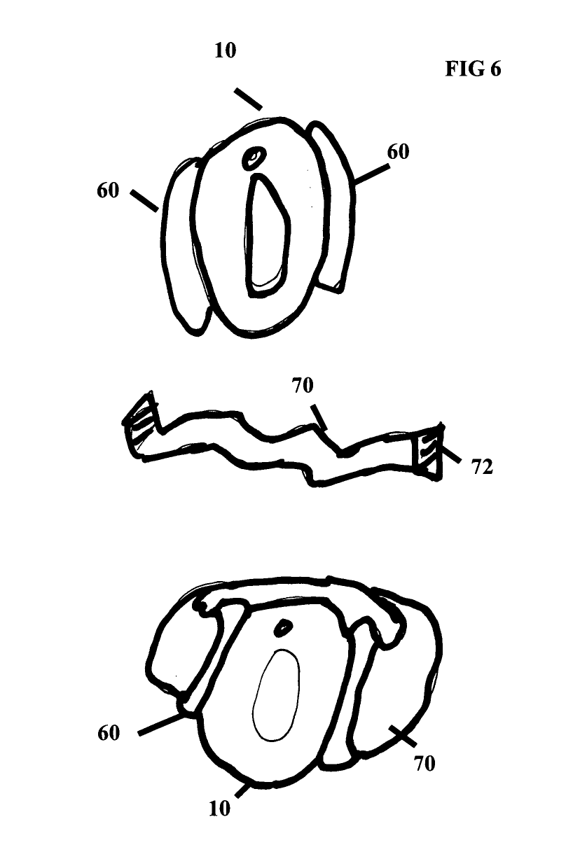

FIG. 6 is a diagram showing the second way to attach unit to the body. Embodiment (10) has two molded brackets (60) these brackets hold a strap (70) with Velcro tips (72) which can wrap around (60) and secure the unit to an individual's body part. The strap (70) gives the user the added benefit of increasing the vibration waves and/or mechanical muscular manipulation through the body because it allows the user to apply pressure.

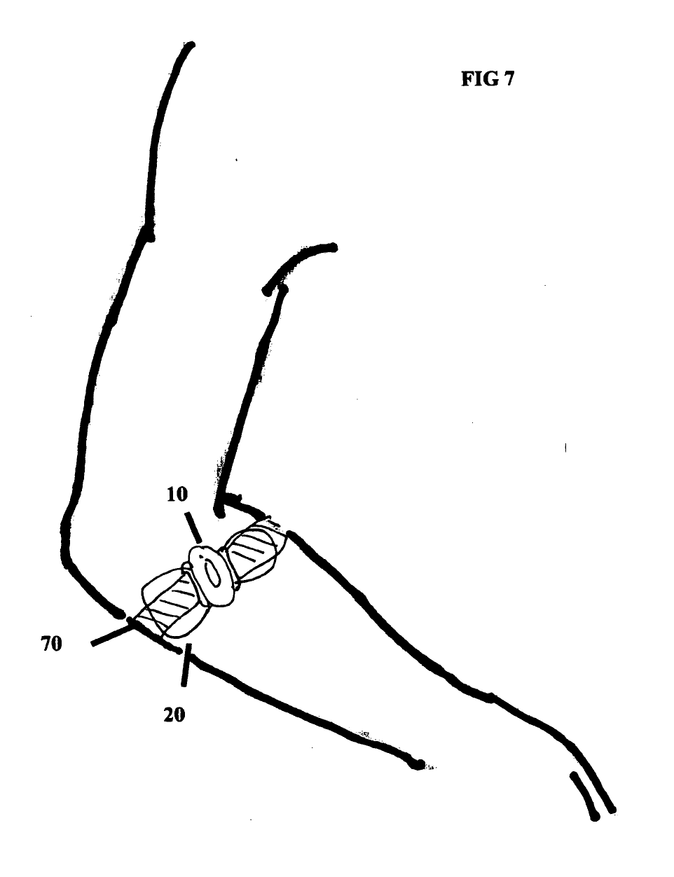

FIG. 7 is a diagram of an arm showing the unit (10) with embodiment (70) attached to someone's arm. Unit is shown with (20) electrode attached to body with strap (70) over the top of (20) to create more pressure and increase the amount of vibration and/or mechanical muscular manipulation a user can feel and absorb.

The treatment protocol employing the embodiment described in FIGS. 1 through 7 can be used for example, to treat carpel tunnel in the forearm and hands, planter fasciitis in the foot, or back pain. It is believed this operates, at least in part, by stimulating cell activities producing an increased ATP concentration. In this specific treatment protocol, a patient will first prepare the appropriate skin area by careful washing and drying to remove skin oils, cosmetics, or other foreign materials from the skin surface. A patient will connect the electrode pads (20) to the base unit (10), and if desired, the attach strap (70) over the electrode pads (20) ordinarily have a sticky surface so that they will adhere to the skin. If the micro current nerve stimulation device (10) is not already turned on, the control switch (14) will be turned to on and operated to a set of instructions provided with the unit. Only current in a predetermined micro amperage is applied.

The micro current nerve stimulation device (10) is programmed to deliver 20 minutes of treatment per treatment. The micro current nerve stimulation device (10) is also programmed to audibly notify a user as the treatment proceeds at each stage of the treatment. During the treatment the micro current nerve stimulation device (10) will reverse the polarity of the electrodes every two seconds. The treatment is ordinarily administered twice a day—once in the morning and once in the evening. It has been found in practice that stimulating with a square wave form for the intervals and frequencies described above is an effective treatment for carpel tunnel syndrome, planter fasciitis in the foot, or back pain.

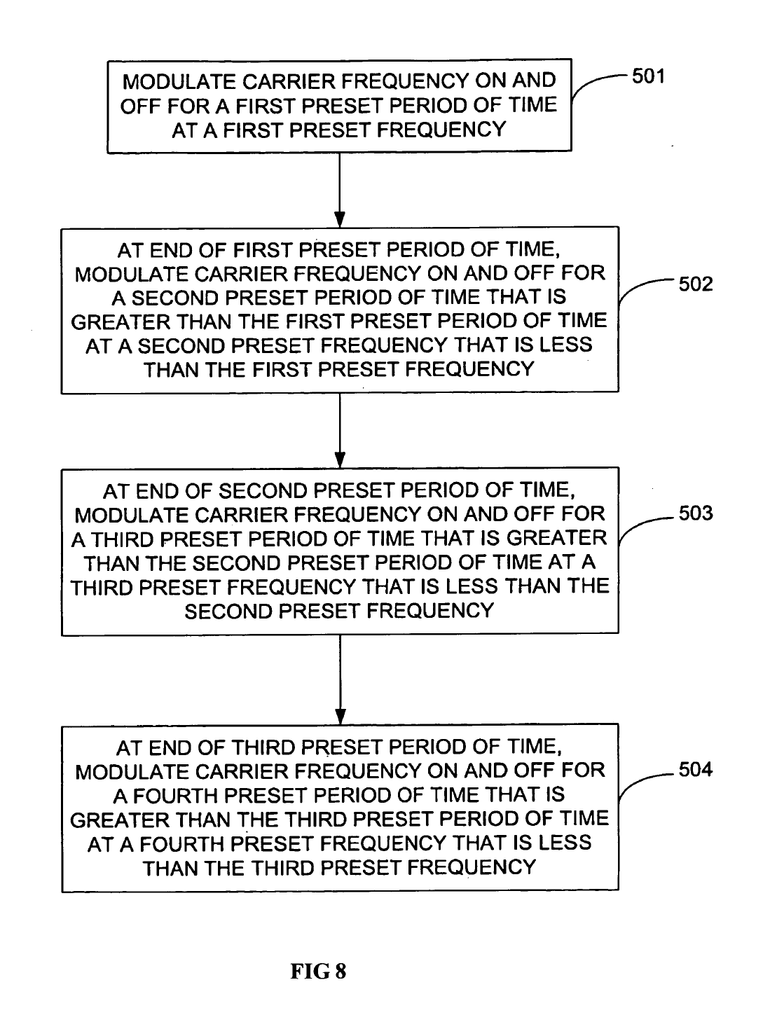

FIG. 8 shows a treatment progression (501) the user presets their desired mode. The unit operates for the preset period (502) after the first period a second period is employed (503) and a third and final mode is operated in (504) all this transpires within one twenty minute session.

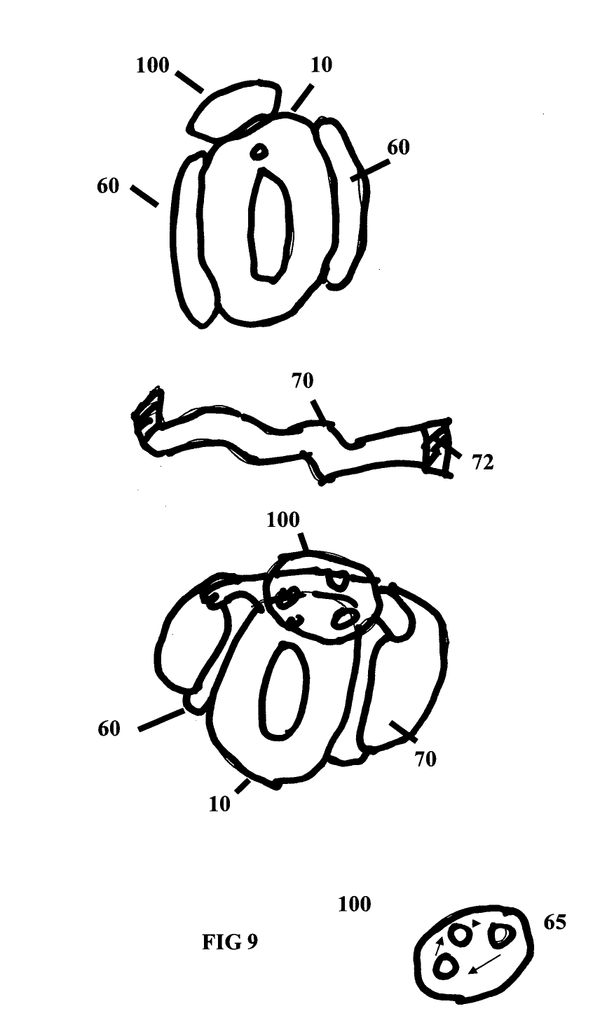

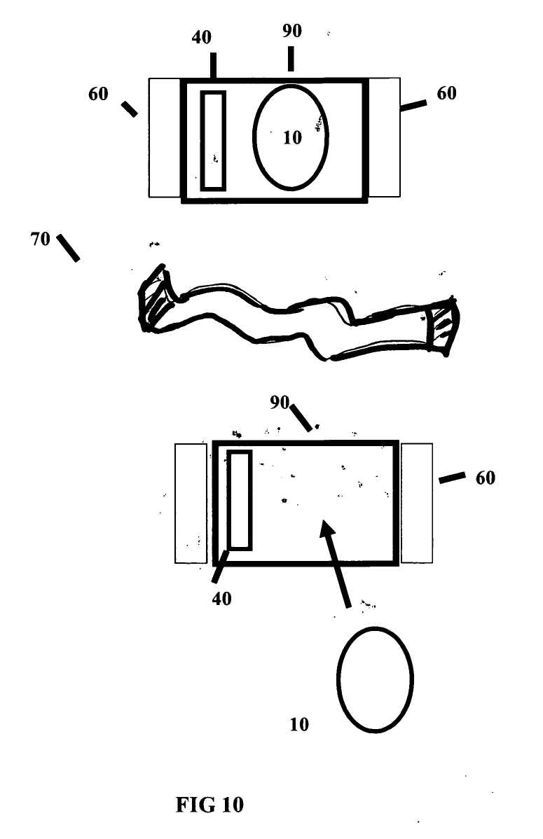

FIG. 9 shows the micro current stimulation device with a mechanical muscular manipulation component. The manual manipulation version can be used in conjunction with the electrotherapy and the vibration. The muscular manipulation component is made into the base unit (10) and is illustrated as (100) with in this small area there are 3 protruding nodes (65) that spin in a clockwise direction at 7 to 10 revolutions per minute. Base unit (10) includes open area for strap to attach (60) which strap (70) can slide through and hold the unit so user can strap it to their arm, foot or back (wrapping the strap (70) around their waist or shoulders. Inside base unit (10) there includes a vibration box (40) on FIG. 3 and muscular manipulation component (100) all the units can be operated independent and dependant on each other. To use electronic and vibration they are detailed in previous FIGS. 1 through 8. Electrode (22) connects by means of snap pin (20) to electrode jacks located on the back of component (10). An on/off switch is (14) on the front of the box (10). Embodiment (16) is an illuminator led light, detailed in FIG. 1 which shows when the unit is operating for the user. Electrode (20) will be applied to a user completing an electrical circuit which allows a micro current to pass from one electrode through the body of the user to the other electrode to complete the circuit. The user can connect to the arm, foot or back with strap (70) through (60) the hooks protruding from the base unit (10). Velcro is used to secure strap (72) FIG. 10 shows another way to attach the base unit with the micro current stimulation device a second way to attach to the body, base unit only (10) snaps into base unit (90) which permanently houses the vibration unit alone. Inside (90), there is a vibration box (40), base unit (10) can snap into (90) so the user can remove the microprocessor and use with just the vibration (40) or use together in combination base (10). Connected to the box (10) are is an electrode pad (22) which is discussed in detail in the previous FIGS. 1 through 9. The base unit (10) connects to (90) which can be attached to strap (70) and attached to the users arm or other parts of the body like feet or the back. If the polarity of the current is reversed as part of a treatment method, then more than two electrodes may be employed. As discussed in previous FIGS. 1 through 8 in more detail electrode (22) connects by means of snap pin (20) to electrode jacks located on the back of component (10). An on/off switch is (14) on the front of the box (10).

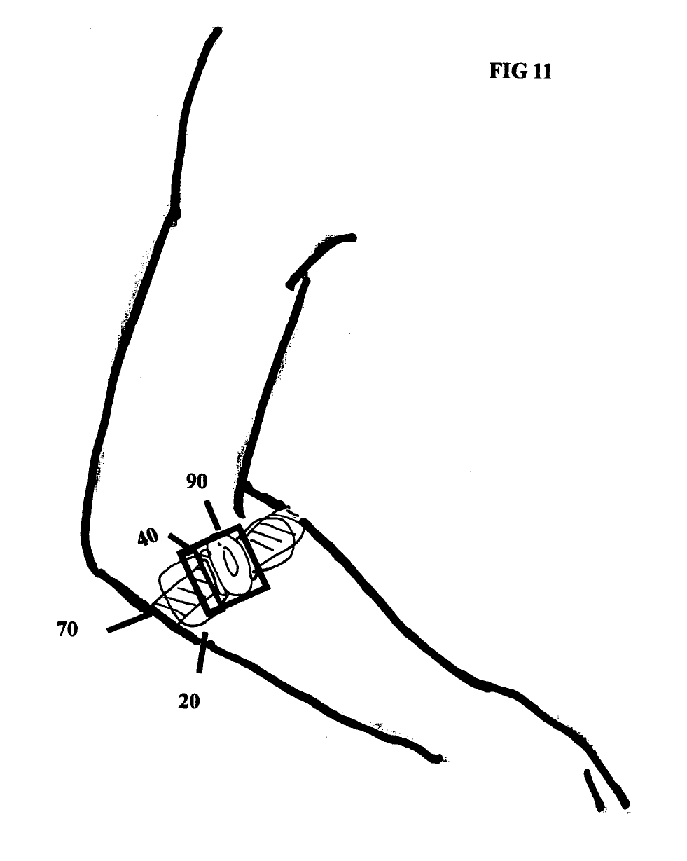

FIG. 11 shows base unit (90) with base unit (10) that snaps into base unit (90) and strap (70) attached to a users arm with sticky pad (20). Base unit (90) with vibration unit (40).

1. A method for applying micro current electrical stimulation to a body, the method comprising the steps of:
(a) attaching at least an electrode in proximity to a specific body part and attaching the base unit to sticky electrode pad on the body at a point away from said specific body part;
(b) applying a micro current across said first and said second electrode wherein said micro current is greater than 900 microampere and less than 1 milliampere;
(c) using a direct current carrier signal for said micro current, said carrier signal frequency from 10,000 Hz up to 20,000 Hz; and
(d) modulating said carrier signal on and off at least first, second and third preset frequencies during at least first, second and third preset time periods, respectively, each of said preset frequencies being lower than said carrier signal frequency, the first preset frequency being higher than the second preset frequency, the second preset frequency being higher than the third preset frequency, the third preset time period being greater than the second preset time period, the second preset time period being greater than the first preset time period, the second preset time period beginning when said first preset time period ends, said third preset time period beginning when said second preset time period ends.
(e) using vibration at 1800 RPM's at the same time as the electrical stimulate. Vibration is a small motor mounted in the base unit.
(f) and/or using the mechanical muscular manipulation portion to disperse the accruing lactic acid from muscles

2. The method of claim 1 wherein said step of modulating said carrier signal on and off further includes applying said preset frequencies in a square wave form combined with vibration and/or mechanical muscular manipulation

3. A method for applying micro current electrical stimulation to a body, the method comprising the steps of:
(a) attaching at least an electrode in proximity to a specific body part and attaching the base unit to sticky electrode pad on the body at a point away from said specific body part;
(b) Having a base unit with openings on the side that a strap can attach to and hold base unit tighter against the body and increasing the intensity of the vibration and/or the mechanical muscular manipulation.
(c) applying a micro current across said first and said second electrode wherein said micro current is greater than 900 microampere and less than 1 millampere;
(d) using a direct current carrier signal for said micro current, said carrier signal frequency from 10,000 Hz up to 20,000 Hz; and
(e) modulating said carrier signal on and off at least first, second and third preset frequencies during at least first, second and third preset time periods, respectively, each of said preset frequencies being lower than said carrier signal frequency, the first preset frequency being higher than the second preset frequency, the second preset frequency being higher than the third preset frequency, the third preset time period being greater than the second preset time period, the second preset time period being greater than the first preset time period, the second preset time period beginning when said first preset time period ends, said third preset time period beginning when said second preset time period ends.
(f) using vibration at 1800 RPM's at the same time as the electrical stimulate. Vibration is a small motor mounted in the base unit.
(g) and/or using the mechanical muscular manipulation portion at the same time, such as a small spinning attachment that spins in a circular motion with nodes protruding out, to disperse the accruing lactic acid from muscles.

4. The method of claims 1, 2 and 3 wherein said step of reversing the polarity of said micro current occurs at an interval of two seconds.

5. A method for applying micro current electrical stimulation to a body, the method comprising the steps of:
(a) attaching at least an electrode in proximity to a specific body part and attaching the base unit to sticky electrode pad on the body at a point away from said specific body part;
(b) applying a micro current across said first and said second electrode wherein said micro current is greater than 900 microampere and less than 1 millampere;
(c) using a direct current carrier signal for said micro current, said carrier signal frequency from 10,000 Hz up to 20,000 Hz; and
(d) modulating said carrier signal on and off at least first, second and third preset frequencies during at least first, second and third preset time periods, respectively, each of said preset frequencies being lower than said carrier signal frequency, the first preset frequency being higher than the second preset frequency, the second preset frequency being higher than the third preset frequency, the third preset time period being greater than the second preset time period, the second preset time period being greater than the first preset time period, the second preset time period beginning when said first preset time period ends, said third preset time period beginning when said second preset time period ends.
(e) using vibration at 1800 RPM's at the same time as the electrical stimulate. Vibration is a small motor mounted in the base unit.
(f) and/or using the mechanical muscular manipulation portion at the same time
(g) having a base unit separate from the controller unit where as the controller unit can snap into a unit that has vibration and/or mechanical muscular manipulation built into it and can attach to the arm or other body part with a strap.

6. A method for applying micro current electrical stimulation to a body, the method comprising the steps of:
(a) attaching at least an electrode in proximity to a specific body part and attaching the base unit to sticky electrode pad on the body at a point away from said specific body part;
(b) applying a micro current across said first and said second electrode wherein said micro current is greater than 900 microampere and less than 1 millampere;
(c) using a direct current carrier signal for said micro current, said carrier signal frequency from 10,000 Hz up to 20,000 Hz; and
(d) modulating said carrier signal on and off at least first, second and third preset frequencies during at least first, second and third preset time periods, respectively, each of said preset frequencies being lower than said carrier signal frequency, the first preset frequency being higher than the second preset frequency, the second preset frequency being higher than the third preset frequency, the third preset time period being greater than the second preset time period, the second preset time period being greater than the first preset time period, the second preset time period beginning when said first preset time period ends, said third preset time period beginning when said second preset time period ends.
(e) using mechanical vibration at 7 to 10 times per minute at the same time as the electrical stimulate. Mechanical manipulation includes protruding nodes that that spin within the base of the unit and against the user's skin.
(f) and/or using the mechanical muscular manipulation portion to disperse the accruing lactic acid from muscles
